(19) United States
(12) Patent Application Publication
(10) Pub. No.: US 2022/0096215 A1
(43) Pub. Date: Mar. 31, 2022

(54) Process For The Preparation Of A Dental Shaped Body

(71) Applicant: Ivoclar Vivadent AG, Schaan (LI)

(72) Inventors: Markus Rampf, Seewis Dorf (CH); Nina Maria Mathis, Ruggel (LI); Christian Ritzberger, Grabs (CH)

(21) Appl. No.: 17/472,755

(22) Filed: Sep. 13, 2021

(30) Foreign Application Priority Data
Sep. 30, 2020 (EP) 20199437.3

(51) Int. Cl. A61C 13/08; A61C 13/00; A61C 13/083; B28B 1/14

(52) U.S. Cl. CPC A61C 13/082; A61C 13/0022; B33Y 80/00; A61C 13/081; B28B 1/14; A61C 13/083

(57) ABSTRACT

A process for the production of a dental shaped body, such as a dental blank or a dental restoration, in which a suspension of a zirconium oxide starting material is gelled by means of a gelling agent, and the use of a suspension of a zirconium oxide starting material as dental material, the suspension containing a gelling agent.

### CROSS-REFERENCE TO RELATED APPLICATIONS

This application claims priority to European Patent Application No. 20199437.3 filed on Sep. 30, 2020, the disclosure of which is incorporated herein by reference in its entirety.

### TECHNICAL FIELD

The invention relates to a process for the preparation of a dental shaped body and in particular for the preparation of dental blanks or dental restorations.

### BACKGROUND

Oxide ceramics, such as zirconium oxide ceramics, are widely used in the manufacture of dental restorations due to their advantageous mechanical properties. Especially zirconia materials based on tetragonal polycrystals stabilized with Y₂O₃ are suitable as starting materials for dental restorations due to their advantageous mechanical properties.

Zirconium oxide ceramics can be produced using various processes. Preferred processes are (i) axial pressing or cold isostatic pressing (CIP) followed by conventional sintering, (ii) slip casting followed by conventional sintering and (iii) hot pressing (HP), hot isostatic pressing (HIP) or spark plasma sintering (SPS). The production of dental restorations made of zirconia, as also described in WO 2018/115529 A1 and corresponding US 20190381769, which U.S. published application is hereby incorporated by reference in its entirety, typically involves two thermal densification steps separated by a shaping step. The zirconia starting materials are typically first cast or mechanically densified under pressure and then presintered to an intermediate open-pored state to produce a blank. This blank is suitable for shaping or pre-shaping, e.g. by machining in a CAD/CAM process. The shaped blank can then be subjected to a final thermal densification in a further sintering step.

Another approach for the production of dental zirconia restorations uses additive manufacturing processes, such as the 3D inkjet printing process described in EP 3 659 547 A1 and corresponding US 20200171699, which U.S. published application is hereby incorporated by reference in its entirety. This process typically produces a three-dimensional body by layering suitable suspensions on top of each other in a printing process and then sintering them.

When preparing dental restorations, it is desirable to imitate the complex coloration of natural teeth with color gradients and 3D color effects. This poses a great challenge, especially for dental restorations made of zirconium oxide, which is why other materials, such as lithium silicate glass-ceramics, are preferred, especially in the anterior region, despite their mechanical properties often being inferior to those of zirconium oxide. A number of possibilities are known for the coloration of dental restorations made of zirconium oxide.

By using differently colored suspensions, additive manufacturing processes generally also allow the fabrication of dental zirconium oxide restorations with complex coloring. However, since such additive processes are very complex, procedures with a machining shaping step are generally preferred.

Another common approach to color dental zirconia restorations uses infiltration of the ceramic material in the porous state with colored metal compounds. This usually involves drying the body to be colored, infiltrating all or part of it with a coloring solution, cleaning and drying the surface, repeating the process with additional coloring solutions if necessary, and finally sintering the colored body.

In another approach, colored zirconia powders are first prepared by coprecipitating zirconia together with coloring elements or by contacting a zirconia powder with solutions of coloring compounds. Such colored zirconia powders can be layered on top of each other in powder form or in suspension form, for example, to fabricate multicolored dental restorations. The disadvantage of this method, however, is that uncontrollable mixing effects occur at the interface of the layers. In addition, only predominantly even layer arrangements can be achieved, which prevents the creation of complex three-dimensional colorations in which, for example, the core of the restoration has a different color than the shell around the core.

US 2015/0282905 A1, which is hereby incorporated by reference in its entirety, discloses a process for the manufacture of dental zirconia blanks in which zirconia suspensions containing a polymerizable component such as acrylate or methacrylate are polymerized by means of a heat or radiation treatment, resulting in suspensions with increased viscosity. Two or more zirconia suspensions with solids contents of only up to 35% by weight are used, which differ with respect to the zirconia crystal phases, which is why the blank produced has areas with different mechanical properties, especially hardness, and different translucency. In order to achieve coloration or multi-color, the blanks must be treated with coloring solutions. The process is unsuitable for the use of commercial zirconium oxide powders because this leads to limitations in the machinability of blanks and to disadvantageous optical properties.

### SUMMARY

The invention is based on the problem of providing a process for the preparation of dental shaped bodies which is efficient, allows easy shaping and is also suitable for the preparation of multicolored shaped bodies. The process should also be associated with low shrinkage and in particular allow the production of complex colorations.

Surprisingly, this problem is solved by the process for the preparation of a dental shaped body according to the claims. The invention further relates to the dental shaped body according to and the use of a suspension of a zirconium oxide starting material according to the claims.

### BRIEF DESCRIPTION OF THE DRAWINGS

In the drawings:

FIG. 1 shows cross sections through presintered test specimens;

FIG. 2 shows a scanning electron microscope image of an interface between two different suspensions after dense sintering;

FIG. 3 shows test specimens after determining the biaxial strength; and

FIG. 4 shows dried green bodies, presintered bodies and densely sintered bodies.

### DETAILED DESCRIPTION

The process according to the invention for the preparation of a dental shaped body is characterized in that a suspension of a zirconium oxide starting material is gelled, wherein the suspension comprises a gelling agent.

Surprisingly, it has been found that the process according to the invention allows for the simple production of dental shaped bodies even from commercially available zirconium oxide powders. Surprisingly, blanks made from commercial zirconia powders can also be easily machined, and dental restorations prepared from commercially available zirconia powders using the process according to the invention have surprisingly advantageous optical properties.

It has also been surprisingly shown that the process allows an easy shaping and allows the production of various dental shaped bodies with high quality of the edges and surfaces. Surprisingly, the process also allows an easy handling of the gelled suspension and the production of shaped bodies with thin wall thicknesses. Therefore, the process even allows the preparation of dental restorations in a casting process, where a shaping by machining can be completely or partially dispensed with.

Surprisingly, it has also been found that the process also offers advantages for the production of multicolored dental shaped bodies, as it avoids mixing effects that typically occur at the interface of two layers. Surprisingly, the process also allows the production of multicolored dental shaped bodies with complex, even three-dimensional colorations and manifold layer arrangements. Furthermore, it has been found that the microstructure of dental restorations with several different layers exhibits a surprisingly high strength even at the interfaces.

The terms “color” and “colored”, within the meaning of the invention, refer to the color value, brightness and/or translucency, in particular to the color value and/or brightness.

Color values and brightness can be determined by the L*a*b value, in particular according to DIN EN ISO 11664-4, or according to a shade guide commonly used in the dental industry. The color measurement can be performed with commercially available measuring instruments, such as a spectrophotometer CM-3700d (Konica Minolta). Examples of shade guides are the Vitapan Classical® and the Vita 3D Master®, both from VITA Zahnfabrik H. Rauter GmbH & Co KG, and the Chromascop® from Ivoclar Vivadent AG.

Translucency is the light transmission of a material, i.e. the ratio of transmitted to incident light intensity. Translucency can be determined in terms of the contrast value (CR value) according to British Standard 5612.

Two suspensions exhibit different colors within the meaning of the present invention if they or densely sintered dental shaped bodies made therefrom differ with respect to the L*a*b values, the color values by means of shade guides and/or the translucency, in particular the L*a*b values and/or the color values.

The suspension used in the process according to the invention comprises a gelling agent. For the purposes of the present invention, a gelling agent is an agent by means of which the suspension can be brought into a non-flowable state, preferably a viscoelastic state. For the purposes of the present invention, “visco-elastic” are suspensions which are not flowable and also have elastic properties. The transformation of the suspension into a non-flowable, preferably visco-elastic state is called “gelling” and the material obtained is called “gel” or “gelled suspension”.

In an embodiment of the process according to the invention, the gelled suspension is viscoelastic and preferably demoldable. A gelled suspension is “demoldable” within the meaning of the invention if it can be removed from a mold in such a way that its outer shape after removal is essentially a counterpart of the mold used.

In a preferred embodiment, the gelled suspension is characterized by the fact that in a needle penetration test with a penetrometer (e.g. PNR 10 from SUR Berlin) using a drop stick weighing 15 g and a round stainless steel pin needle weighing 3 g at a load time of 0.2 s, the pin needle penetrates the suspension, but not more than 20 mm, the pin needle having a diameter of 3 mm, a total length of 58 mm and a straight conical tip with a height of 5.6 mm and an opening angle of 30°. Suitable is e.g. the pen needle type 18-0222 from Petrotest GmbH. The suspension is either divided or removed from a mold immediately before the test is performed and then the state of the suspension is determined in an area that was previously inside the suspension or in contact with the mold.

The zirconium oxide starting material is usually present as suspension in a liquid medium. The liquid medium may comprise inorganic and/or organic solvents. A preferred inorganic solvent is water. Preferred organic solvents are water-miscible organic solvents, in particular alcohols, ketones, esters, ethers and mixtures thereof. Mixtures of water with one or more organic solvents can also be used. The liquid medium comprises in particular water. Especially preferred the liquid medium consists essentially or exclusively of water. The liquid medium may also comprise liquid additives.

The solid component of the suspension consists of the zirconium oxide starting material and optionally solid additives, wherein the zirconium oxide starting material preferably makes up at least 80 wt.-%, in particular at least 90 wt.-% and particularly preferably at least 95 wt.-%, based on the weight of the solid component.

In a preferred embodiment, the suspension comprises 55 to 70 wt.-%, preferably 60 to 70 wt.-%, and especially preferred 65 to 70 wt.-% zirconium oxide starting material.

Surprisingly, it has been found that the use of suspensions with a high zirconium oxide content allows the production of dental shaped bodies, wherein the shrinkage typically occurring during drying and sintering is lower than in processes using suspensions with lower solids contents. Surprisingly, the high content of zirconium oxide has also been shown to allow a more efficient preparation of dental shaped bodies, as the gelling step and a drying step can be shortened.

The zirconium oxide starting material is usually a particulate zirconium oxide, preferably a zirconia powder. The zirconium oxide powder preferably consists of zirconium oxide particles, which have a d₅₀ particle size, based on the volume of the powder, of 50 to 250 nm, in particular 60 to 250 nm, especially preferably 80 to 250 nm. It is also preferred that the zirconium oxide powder has a primary particle size of 30 to 100 nm, determined by dynamic light scattering (DLS).

The zirconium oxide starting material may be based on unstabilized or partially stabilized monoclinic zirconia, partially stabilized tetragonal zirconia or fully stabilized cubic zirconia or mixtures thereof, in particular on unstabilized or partially stabilized monoclinic zirconia or partially stabilized tetragonal zirconia or mixtures thereof. Furthermore, the zirconia starting material is preferably polycrystalline and, particularly preferred, essentially polycrystalline tetragonal zirconia (TZP).

In another preferred embodiment, the zirconium oxide starting material comprises Y₂O₃, La₂O₃, CeO₂, MgO and/or CaO, preferably in an amount of 2 to 14 mol %, in particular 2 to 8 mol %, especially preferably 3 to 5 mol %-, based on the zirconia content. Particularly preferably the zirconium oxide starting material comprises Y₂O₃.

The zirconium oxide starting material used in the process according to the invention may also be colored. The desired coloration can be achieved by coprecipitating the zirconium oxide starting material with one or more coloring elements. This coloration is also called doping and is usually performed during the production of the zirconium oxide starting material. Suitable coloring elements are, for example, Fe, Mn, Cr, Ni, Ti, Co, Pr, Ce, Eu, Gd, Nd, V, Yb, Ce, Tb, Er and Bi. The zirconium oxide starting material has particularly preferred a color that corresponds to the color of the natural tooth material.

The suspension used in the process according to the invention also comprises a gelling agent. The gelling agent can be present in the suspension in the solid component and/or in the liquid medium. It may also depend on the ambient conditions, in particular the temperature, whether the gelling agent is in a solid or liquid state.

Preferably, the gelation of the suspension can be initiated and/or controlled by the influence of one or more parameters, which within the meaning of the invention are called gelation parameters. Suitable gelation parameters are a change in temperature or pH of the suspension, an incubation period and irradiation. Preferably, gelation can be initiated and/or controlled by a change in temperature of the suspension.

In a particularly preferred embodiment of the process according to the invention, the suspension is gelled by cooling. The temperature before cooling is preferably in the range of 50° C. to 100° C., in particular 60° C. to 80° C. The temperature after cooling is preferably in the range of −10° C. to 40° C., preferably 0° C. to 25° C.

It is preferred that the gelling agent used in the process according to the invention is a polysaccharide, a protein or a mixture thereof, preferably agarose, agar, alginate, gellan, carrageenan, pectin, chitosan, chitin, gelatin or a mixture thereof, and particularly preferably agarose.

Gelling agents that are not of natural origin, such as an isobutyl/maleic anhydride copolymer, can also be used.

The suspension preferably comprises at least 0.01 wt.-%, in particular 0.01 to 1.5 wt.-%, more preferably 0.02 to 1 wt.-% and particularly preferably 0.03 to 0.5 wt.-% of gelling agent.

In a preferred embodiment, the suspension comprises at least 0.03 wt.-%, in particular 0.03 to 0.5 wt.-%, and particularly preferably 0.03 to 0.4 wt.-% of agarose.

In another preferred embodiment, the suspension comprises at least 0.1 wt.-%, in particular 0.1 to 1 wt.-% and particularly preferably 0.1 to 0.8 wt.-% agarose, based on the weight of the liquid medium.

The suspension may comprise solid and/or liquid additives in addition to the zirconium oxide starting material, the gelling agent and the liquid medium. For example, the suspension may comprise dispersing agents, binders, pH adjusting agents, stabilizers and/or defoamers, as are described in EP 3 659 547 A1.

In a preferred embodiment, the suspension comprises defoamer to prevent air bubbles. The defoamer or defoamers are typically used in an amount of 0.001 to 1 wt.-%, preferably 0.001 to 0.5 wt.-% and particularly preferably 0.001 to 0.1 wt.-% in the liquid medium, based on the amount of solid in the suspension. Examples of suitable defoamers are paraffin, silicone oils, alkylpolysiloxanes, higher alcohols, propylene glycol, ethylene-oxide-propylene oxide adducts and in particular alkylpolyalkyleneglycol ethers.

It is preferable that the suspension comprises, in addition to the gelling agent, not more than 2 wt.-%, preferably not more than 1 wt.-%, particularly preferably not more than 0.55 wt.-% of organic components. Organic components include organic solvents and solid and liquid organic additives.

There are several ways to provide the suspension of the zirconium oxide starting material containing the gelling agent. For example, the zirconium oxide starting material can be mixed with the gelling agent and the resulting mixture suspended in the liquid medium. It is also possible to add the gelling agent in solid or liquid state to the suspension of a zirconium oxide starting material.

In a preferred embodiment, first the gelling agent, e.g. agarose, is added to the suspension as a solid and then the suspension with the solid gelling agent is heated to a temperature at which the gelling agent changes into a liquid state.

In another preferred embodiment, the gelling agent is brought to a liquid state by heating and then added to the suspension, the suspension preferably being at a temperature at which the gelling agent remains in the liquid state.

When using agarose as a gelling agent, agarose, for example, can first be placed in a liquid medium and heated to a temperature above the melting point of agarose, e.g. to above 88° C. This medium with agarose can then be added to the suspension, the suspension having a temperature above the temperature at which the agarose gels, e.g. 70° C.

The process according to the invention may comprise gelling at least two suspensions with different compositions, wherein the suspensions or the densely sintered bodies produced therefrom preferably have different colors, in particular different color values and/or brightness.

The different colors of the suspensions are usually achieved by the suspensions differing in the type and/or amount of coloring elements.

It is preferred that the at least two suspensions with different compositions are brought into direct contact with each other. This is usually done by first at least partially gelling a first suspension and then applying the second suspension to the first and gelling it as well. It is preferred to cool the first suspension for at least 1 min, preferably at least 3 min and especially preferred at least 5 min, at ambient conditions before the second suspension is applied to the first. In a preferred embodiment, the at least partially gelled first suspension is partially dried before the second suspension is applied.

By using suspensions with different colors, it is possible to produce multicolored dental shaped bodies that mimic the complex coloration of natural teeth. For example, suspensions with different colors can be placed on top of each other to create color gradients.

Complex colorations, such as three-dimensional color gradients, free layer arrangements in which the layers are not even, and core-shell arrangements, can also be generated by the process according to the invention. As a rule, these complex colorations are produced by gelling a first suspension with a gelling agent in the desired shape before a further suspension is applied and gelled. The first suspension can be formed into the desired shape using a mold and/or can be shaped as required before applying the further suspension.

In a preferred embodiment, two differently colored suspensions are layered on top of each other, so that they form an inclined interface. The interface can be essentially straight or curved. Preferred courses of the inclined interface in the shaped body are described in WO 2020/025795 A1 and corresponding US 20210196437, which U.S. published application is hereby incorporated by reference in its entirety. For example, it is preferred that the interface has in a sectional plane through the shaped body running parallel to the insertion axis an angle of 10 to 70°, preferably 10 to 60°, to the axis of rotation of the shaped body and/or has a mamelon structure.

Surprisingly, it has been found that dental shaped bodies can be produced from different suspensions by the process according to the invention, wherein the suspensions in the shaped body form a solid bond and the bond meets the highest mechanical requirements. For example, it was possible to demonstrate the high structural quality at interfaces, and it was also proven that shaped bodies produced according to the process of the invention do not break along the interfaces in material tests.

In an embodiment, the suspension used in the process according to the invention has a viscosity of up to 25 Pas before gelation, in particular up to 10 Pas, particularly preferably 0.001 to 10 Pas. Unless otherwise specified, the viscosity is measured with a rotational viscometer with plate-cone system, diameter 50 mm and angle 1° (MCR302-Modular Compact Rheometer, Anton Paar GmbH) at a shear rate in the range of 0.1 to 5000 s⁻¹ and a temperature below 100° C.

It was found that the viscosity before gelation is usually dependent on the amount of solid component in the suspension, especially the amount of zirconium oxide starting material used, as well as the amount of gelling agent used. It was also found that a higher content of zirconium oxide starting material and/or gelling agent generally leads to a higher viscosity before gelling.

In a preferred embodiment of the process, the suspension is placed in a mold. It is preferred that gelation of the suspension takes place at least partially in the mold. The suspension can be added to the mold, for example, by casting or injection. The suspension can also be added to the mold through one or more casting channels.

In another preferred embodiment, different suspensions are placed in a mold. The different suspensions can be added to the mold simultaneously or one after the other.

The mold preferably corresponds to a negative shape of the dental shaped body to be produced by the process according to the invention. It is particularly preferred that the mold corresponds to an enlarged negative shape of the dental shaped body, the degree of enlargement being adapted to the shrinkage occurring during further processing.

It has been shown that in the process according to the invention, the time course of the gelation of the suspension, i.e. the gelation process, can usually also be controlled by selecting suitable process parameters. Suitable means for controlling the gelation process are in particular the amount of gelling agent used, the solids content of the suspension and the selected gelation parameter. As a rule, the gelation process can be enhanced by a large amount of gelling agent, a high content of zirconium oxide starting material and/or a strong influence of the gelation parameter, such as a strong or rapid temperature change.

When preparing dental shaped bodies from several suspensions, it can be advantageous to adjust the gelation process or the viscosity of a suspension prior to gelation to the shapes to be achieved. The specific choice of the flow properties of the suspensions before the gelation and the control of the gelation process can be helpful to achieve desired arrangements of the suspensions, in particular complicated three-dimensional arrangements.

A gelled suspension which has not yet been subjected to sintering is also referred to as a green body within the meaning of the invention.

In a preferred embodiment of the process, the prepared dental shaped body is a green body.

The green body is preferably characterized by a density of 2.5 to 4.0 g/cm³ and in particular 2.6 to 3.8 g/cm³ and especially preferably 2.8 to 3.6 g/cm³.

The strength of the gelled suspension usually depends on the amount of gelling agent present in the suspension and the content of zirconium oxide starting material. In particular, the strength can usually be increased by using more gelling agent and/or a higher content of zirconium oxide starting material.

In one embodiment of the process, a gelled suspension is divided into two or more green bodies before drying, from which two or more dental shaped bodies can then be prepared. In another embodiment of the process, a gelled suspension is subjected to shaping. The physical properties of the gelled suspension, which has not yet been subjected to a drying step, allow it to be easily divided or processed by mechanical and especially manual methods.

It has been shown that it can be advantageous for the process according to the invention to cool the gelled suspension to a temperature below room temperature. Cooling is usually accompanied by an increase in viscosity/strength, which may result in the gelled suspension being easier to process or remove from a mold. When using a gelling agent that gels by cooling, the gelation process can usually also be enhanced by exposing the suspension to be gelled to a cooled environment. It may be advantageous, for example, to place the suspension to be gelled in a pre-cooled mold to achieve rapid gelation.

In a preferred embodiment, the suspension is cooled down to a temperature of less than 15° C., preferably less than 10° C., especially preferably 1° C. to 8° C.

In a preferred embodiment, the gelled suspension, i.e. the green body, is subjected to drying to prepare a dried green body. Usually the drying of the green body is an incomplete drying, i.e. the content of the liquid medium in the green body is reduced, wherein even after drying liquid medium is still present in the green body. As drying progresses, the green body typically loses its elastic properties, so that a dried green body is usually no longer viscoelastic. If the process involves the use of a mold, the drying of the gelled suspension can take place inside the mold and/or after removal from the mold.

It is preferable that drying be carried out for a period of at least 12 h, and in particular at least 24 h.

Drying can take place under ambient conditions or under controlled conditions, i.e. at controlled temperature and/or humidity. Preferably drying is carried out at a temperature of 1° C. to 99° C., in particular 10° C. to 80° C., especially preferably 20° C. to 50° C., and a relative humidity of 0 to 99%, in particular 1 to 80% and especially preferably 1 to 50%.

The drying of the green body can for example be carried out for 24 h under ambient conditions.

The dried green body is preferably characterized by a density of 2.5 to 4.0 g/cm³, in particular 2.6 to 3.8 g/cm³ and especially preferably 2.8 to 3.6 g/cm³. Furthermore, the dried green body preferably has a moisture content of up to 15 wt.-%, in particular up to 10 wt.-% and particularly preferably up to 5 wt.-%.

As a rule, the drying of the green body is accompanied by shrinkage. Preferably, a linear shrinkage of no more than 20%, in particular 10% to 20%, occurs during the drying of the green body. The linear shrinkage during drying is the percentage reduction in length along an axis that occurs during the drying of the green body.

In a preferred embodiment of the process according to the invention, the green body is sintered, in particular presintered and/or densely sintered.

Pre-sintering is preferably carried out at a temperature of 800° C. to 1300° C., in particular 850° C. to 1200° C., even more preferably for 900° C. to 1150° C. for in particular 1 to 4 h, preferably 1.5 to 2.5 h.

Densely sintering is preferably carried out at a temperature of 1200° C. to 1600° C., in particular 1300° C. to 1550° C. and especially preferred at 1350° C. to 1500° C. for in particular 5 min to 2 h, preferably for 10 to 60 min and especially preferred for 10 to 30 min.

In a preferred embodiment, the debinding of the green body takes place during sintering. Debinding means the burning out of the organic components, in particular the gelling agents and binders. In a further embodiment, debinding takes place in a separate process step.

As a rule, not only drying but also sintering is accompanied by shrinkage of the green body. It is preferable that the linear shrinkage of the gelled suspension during drying and sintering does not exceed 40%, in particular 20 to 40%, especially preferably 30 to 38%. The linear shrinkage can be determined using a Netzsch dilatometer DIL402 Supreme in a temperature range from 20° C. to 1550° C. at a heating rate of 2 K/min on test specimens.

If the process involves the use of different suspensions, it can be advantageous to match the compositions of the suspensions with respect to shrinkage, in particular shrinkage occurring during sintering.

It is preferred that the dental shaped body prepared by the process according to the invention is a dental blank or a dental restoration and is preferably based on partially stabilized tetragonal, fully stabilized cubic zirconium oxide or mixtures thereof. Typically, the dental restoration comprises essentially no monoclinic zirconia.

The preparation of a dental blank is particularly preferred and includes in an embodiment the pre-sintering of the gelled suspension. Preferably the gelled suspension is dried and then presintered.

In a preferred embodiment, the dental blank has the shape of a rectangular block or of a cylinder.

As a rule, the prepared dental blank is intended to be processed into a dental restoration. For this purpose, the blank is typically machined into the shape of a dental restoration, in particular using a CAD/CAM process. The blank can be attached to a holder, which allows the blank to be inserted into a holder of a CAD/CAM machine intended for this purpose.

In a preferred embodiment, the blank is in one piece with the holder, particularly preferably the blank and the holder are made of the same or different suspensions.

Typically, the dental blank prepared in the process according to the invention is first shaped and then densely sintered, resulting in a dental restoration with the desired properties.

In another particularly preferred embodiment, the dental shaped body prepared by the process according to the invention is a dental restoration. The dental restoration is preferably a bridge, inlay, onlay, veneer, abutment, partial crown, crown or facet.

The dental restoration can, for example, be prepared from a dental blank, the shaping being effected by machining, preferably in a CAD/CAM process.

In another preferred embodiment of the process according to the invention, the preparing of the dental restoration comprises placing the suspension in a mold corresponding to a negative shape of a dental restoration and densely sintering the gelled suspension. The gelled suspension is preferably dried and then densely sintered. In this way, shaping by machining can be avoided or the amount of machining required can be reduced.

It is preferred that a dental restoration prepared according to this embodiment has a minimum wall thickness of 0.12 mm or more.

This embodiment is also particularly suitable for the preparation of multicolored dental restorations with possibly complex three-dimensional color gradients. For this purpose, typically two or more suspensions with different colors are added simultaneously or consecutively into the mold corresponding to the negative shape of the dental restoration to be prepared.

Furthermore, in this embodiment the mold preferably corresponds to an enlarged negative shape of the dental shaped body and the degree of enlargement is adapted to the shrinkage occurring during the process. A particularly precise adapting of the enlargement to the shrinkage is typically possible in processes where the mold is produced using a digital model.

When using a mold that corresponds to a negative shape of the dental restoration, it is preferable that at least part of the mold is produced by an additive process or by machining, especially in a CAD/CAM process. These processes allow a particularly precise design of the mold, taking into account the shrinkage that occurs. As a rule, the mold is made of a material that not only offers sufficient strength but is also inert to the suspension containing the gelling agent.

In another preferred embodiment of the process, which involves the use of a negative shape of the dental restoration, the gelled suspension is presintered and, optionally, subjected to mechanical finishing. The gelled suspension, i.e. the green body, is preferably first dried and then presintered.

If required, the presintered green body can also be subjected to machining, for example to remove material residues caused by casting channels.

It is preferred that a presintered green body is densely sintered after machining and/or finishing.

The invention further relates to a dental shaped body obtainable by the process according to the invention described above. This dental shaped body is in particular multicolored and preferably exhibits at least two different color values and/or brightness values.

Dental shaped bodies prepared according to the process according to the invention are typically characterized by a complex multicolored coloration that cannot be produced by conventional methods.

The dental shaped bodies prepared by the process according to the invention have advantageous mechanical properties.

In a preferred embodiment, the presintered dental shaped body, such as the presintered dental blank, has advantageous properties for machining. It is preferred that this dental shaped body has a Vickers hardness of 300 to 1000 MPa, which is advantageous for a CAD/CAM process. The Vickers hardness can be measured at a force in the range of 2.5 to 5.0 kbf (24.517 to 49.034 N) and in particular at a force of 5.0 kgf (49.034 N) according to ISO 14705:2008.

Furthermore, the presintered shaped body is typically in an open-pored state, wherein the presintered shaped body preferably has a density of at least 30%, in particular 40 to 75% of the density of the densely sintered zirconium oxide ceramic.

A densely sintered dental shaped body prepared according to the process according to the invention, such as a densely sintered dental restoration, preferably has a biaxial strength of at least 500 MPa, in particular at least 600 MPa and particularly preferably at least 800 MPa.

Furthermore, the densely sintered dental shaped body preferably has a density of more than 5.8 g/cm³, in particular more than 5.9 g/cm³ and especially preferably more than 6.0 g/cm³.

The invention also relates to the use of a suspension of a zirconium oxide starting material as dental material, in particular for the preparation of a dental shaped body, wherein the suspension comprises a gelling agent.

All process steps, process parameters and compositional definitions described within the scope of the process according to the invention are also suitable for this use according to the invention.

The invention is explained in more detail in the following on the basis of examples.

### EXAMPLES

### Example 1

A multicolored test specimen was prepared from two different suspensions using the process according to the invention.

For this purpose, two suspensions of zirconium oxide starting material were first prepared. The first suspension included 72 wt.-% of a zirconia powder stabilized with 3 mol % Y₂O₃ (Z-Pex, Tosoh Corporation) and 27.68 wt.-% of water as liquid medium, 0.25 wt.-% dispersing agent (Dolapix® CE64, Zschimmer & Schwarz), 0.06 wt.-% defoamer (Contraspum® K1012, Zschimmer & Schwarz) and 0.01% by weight NH₄OH to adjust the pH value, each based on the total weight of the suspension, and it had a white coloration.

The second suspension included 72 wt.-% of a zirconia powder stabilized with 5 mol % Y₂O₃ (Z-Pex smile, Tosoh Corporation) and 28 wt.-% of water as liquid medium, and it showed a yellow coloration.

Both suspensions were heated to 70° C. An aqueous solution of agarose was prepared, heated to 90° C. and added to the heated suspensions so that the suspensions included 0.2 wt.-% of agarose, based on the heated suspension.

The heated suspensions, to which agarose was added, were then placed in a mold one after the other with a time interval of more than 1 minute.

As soon as the suspensions were poured into the mold, the cooling of the suspensions began. Cooling was additionally supported by storing the mold with the suspensions at 8° C. for 1 hour.

The cooled and during cooling gelled suspensions, which are also called green bodies, were demolded.

The green body was dried for 24 h under ambient conditions and then sintered. A Programatm furnace from Ivoclar Vivadent AG was used for this purpose, as for all the sintering steps mentioned in the examples.

The green body was, for example, presintered at 950° C. for 2 hours.

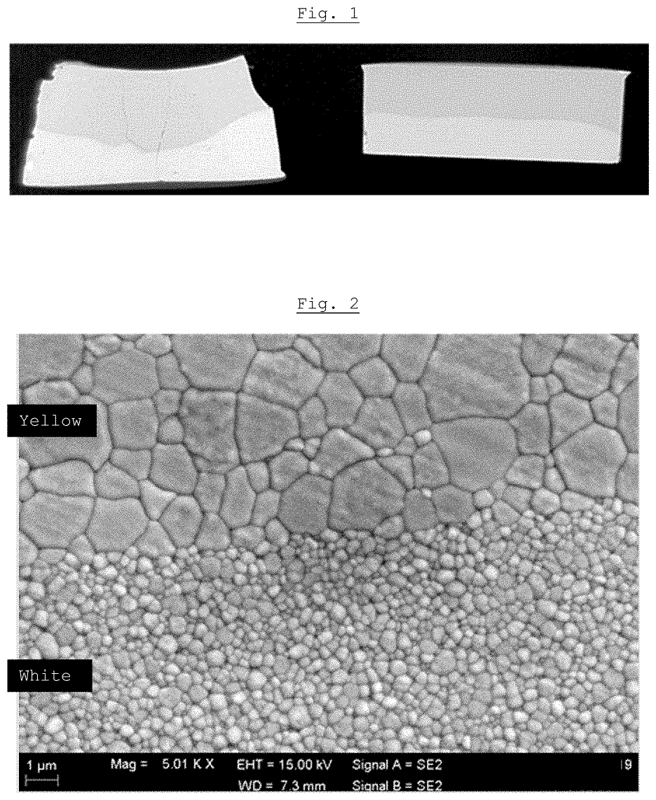

FIG. 1 shows cross sections through presintered test specimens. By using different casting methods, e.g. by varying the casting speed, different layer arrangements could be produced. For example, essentially even (right) and free (left) layer arrangements could be produced. The free layer arrangements showed e.g. inclined planes or wave-shaped layers.

Green bodies were also prepared using the above process, dried and densely sintered at 1500° C. for 25 min.

FIG. 2 shows a scanning electron microscope image of the interface between the two different suspensions after dense sintering. It can be seen that the microstructure at the interface between the yellow (top) and white (bottom) layers has no pores. No pores were also found within the layers of densely sintered zirconium oxide materials.

FIGS. 1 and 2 illustrate that suspensions of different zirconium oxide starting materials comprising gelling agents can be cast over each other without mixing.

### Example 2

Multicolored test specimens for determining the biaxial strength were prepared according to the process of the invention.

Two suspensions were prepared, each including 66.5 wt.-% of zirconium oxide starting material. White Z-Pex smile (Tosoh Corporation) was used to prepare the first suspension and yellow Z-Pex smile yellow (Tosoh Corporation) was used for the second suspension, and the suspensions included Dolapix CE64, Contraspum K1012 and NH₄OH in the amounts indicated for example 1. To the suspensions were each added 0.2 wt.-% of agarose, based on the total weight of the suspension, according to the procedure described for example 1.

Then the first suspension mixed with agarose was first placed in a horizontal cylindrical plastic mold (diameter about 25 mm; length about 30 mm) so that the mold was partially filled. The first suspension was gelled by cooling the plastic mold with the first suspension to 8° C. for about 20 min. Then the second suspension, mixed with agarose, was poured into the mold and gelled by cooling the plastic mold to 8° C. for 1 h.

The gelled cylindrical green body was then demolded and dried for 24 h under ambient conditions. The dried green body was presintered for 2 h at 950° C. and then divided into discs about 2 mm thick. These were then densely sintered at 1500° C. for 25 min and then subjected to a breaking strength test according to ISO 6872.

The densely sintered test specimens had a biaxial strength of 418 to 654 MPa, determined according to ISO 6872.

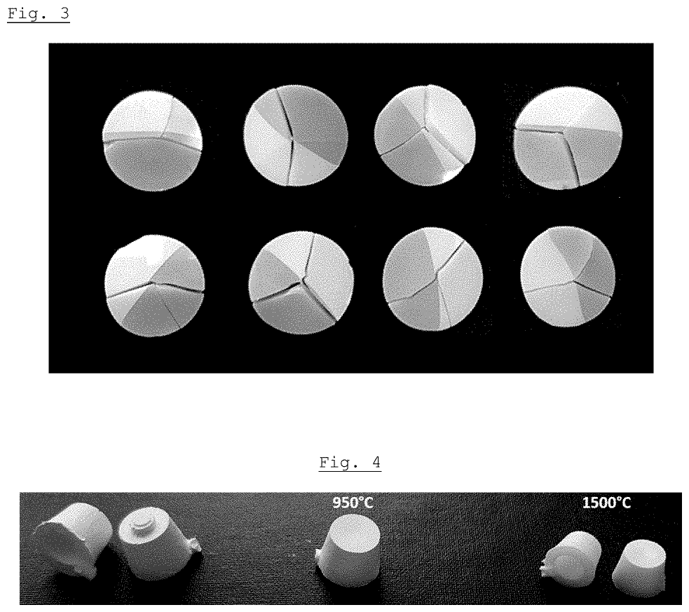

FIG. 3 shows the test specimens after determining the biaxial strength. It can be seen that fracture occurred randomly through the material and not along the interfaces of the different layers. This illustrates that a very high bond strength can be achieved between the layers.

### Examples 3 to 6

The gelling properties of suspensions with the compositions given in Tables 1 and 2 were investigated. Suspensions with different amounts of a zirconium oxide powder stabilized with 5 mol % Y₂O₃ (Z-Pex smile, Tosoh Corporation) in water were prepared. Different amounts of agarose were added to these suspensions according to the procedure described for example 1. For the preparation of example 3, this procedure was changed and the powdered agarose was added directly to the suspension without first preparing an agarose solution. The suspensions containing agarose were placed in conical cylindrical molds and cooled for 1 hour at 8° C. The viscosity of the suspensions before gelation and observations on gelation are given in Tables 1 and 2.

The green bodies were removed from the mold, dried for 24 h at ambient conditions, presintered at 950° C. for 2 h and then densely sintered at 1500° C. for 25 min.

TABLE 1

| Example | 3 | 4 |
|---|---|---|
| Zirconium oxide | 66.5 | 69.8 |
| starting material | | |
| (wt.-%) | | |
| Agarose (wt.-%, | 0.6 | 0.3 |
| based on H₂O) | | |
| Agarose (wt.-%,) | 0.201 | 0.091 |
| based on total | | |
| weight | | |
| Viscosity before | slightly viscous | viscous |
| gelation | | |
| Gelation | Solid green body | Solid green body |
| | after 20 min at 8° C. | after 20 min at 8° C. |
| Density after | 6.01 | 6.011 |
| dense sintering | | |
| (g/cm³) | | |

TABLE 2

| Example | 5 | 6 |
|---|---|---|
| Zirconium oxide | 72.6 | 69.8 |
| starting material | | |
| (wt.-%) | | |
| Agarose (wt.-%, | 0.1 | 0.5 |
| based on H₂O) | | |
| Agarose (wt.-%,) | 0.027 | 0.15 |
| based on total | | |
| weight | | |
| Viscosity before | high viscosity | very high viscosity |
| gelation | | |
| Gelation | Solid green body, | Solid green body |
| | gelation immediately | after 2 min, gelation |
| | after addition | immediately after |
| | of agarose | addition of agarose |

The density of the densely sintered green bodies was determined using the Archimedes method.

The mechanical properties of the shaped body of example 3 were investigated. After drying, the body had a density of 2.71 g/cm³ and its biaxial strength was too low to be determined according to ISO 6872. The residual moisture of the shaped body after drying was determined by step drying up to 200° C. using a halogen moisture meter (HR83, Mettler Toledo) and it was 3.9 wt.-%. After presintering, the shaped body had a density of 2.77 g/cm³ and the biaxial strength according to ISO 6872 was 13 MPa. After dense sintering, the shaped body had a biaxial strength of 595 MPa.

The experiments show that a high content of zirconium oxide starting material or a large amount of agarose in the suspension can increase the viscosity of the suspension before gelation.

Furthermore, the examples show that with a higher content of zirconium oxide starting material, a smaller amount of agarose may be sufficient to prepare a solid green body through gelation.

The examples also show that a larger amount of agarose can result in a higher strength of the green body.

### Examples 7 and 8

For examples 7 and 8 suspensions were prepared according to examples 3 and 4. In addition, stereolithography was used to produce molds, corresponding to the negative shape of a thimble. After addition of the agarose, the suspensions were poured into the molds and cooled for 1 hour at 8° C. The gelled suspensions were demolded and dried for 24 h at ambient conditions.

The dried suspensions, i.e. the dried green bodies, were presintered at 950° C. for 2 hours or densely sintered at 1500° C. for 25 minutes. The density of the densely sintered bodies was 6.03 g/cm³.

FIG. 4 shows dried green bodies (left), presintered (center) and densely sintered (right) bodies of example 7. The bodies shown illustrate that bodies with complicated shapes and thin wall thicknesses can also be obtained.

1. A process for the preparation of a dental shaped body, in which a suspension of a zirconium oxide starting material is gelled, wherein the suspension comprises a gelling agent.

2. The process according to claim 1, in which the suspension comprises 55 to 70 wt.-% of zirconium oxide starting material.

3. The process according to claim 1, in which the zirconium oxide starting material comprises Y₂O₃, La₂O₃, CeO₂, MgO and/or CaO in an amount of 2 to 14 mol %, based on the amount of zirconium oxide.

4. The process according to claim 1, in which the suspension is gelled by cooling.

5. The process according to claim 1, in which the gelling agent is a polysaccharide, a protein or a mixture thereof.

6. The process according to claim 1, in which the suspension comprises 0.03 to 0.5 wt.-% of agarose.

7. The process according to claim 1, in which the suspension comprises, in addition to the gelling agent, not more than 2 wt.-% of organic components.

8. The process according to claim 1, in which at least two suspensions with different compositions are gelled, wherein the suspensions have different colors.

9. The process according to claim 1 in which the suspension is placed in a mold.

10. The process according to claim 1, in which the gelled suspension is viscoelastic and demoldable.

11. The process according to claim 1, in which the suspension is cooled to a temperature of less than 15° C.

12. The process according to claim 1, in which the dental shaped body is a dental blank or a dental restoration and is based on partially stabilized tetragonal, fully stabilized cubic zirconium oxide or mixtures thereof.

13. The process according to claim 12, in which the dental restoration is a bridge, inlay, onlay, veneer, abutment, partial crown, crown or facet.

14. The process according to claim 12, in which the preparation of the dental restoration comprises placing the suspension in a mold corresponding to a negative shape of a dental restoration and densely sintering the gelled suspension.

15. The process according to claim 14, in which at least a part of the mold is produced in an additive process or by machining.

16. The process according to claim 14, in which the gelled suspension is presintered and, optionally, subjected to machining.

17. The process according to claim 12, in which the preparation of the dental blank comprises that the gelled suspension is presintered.

18. The process according to claim 1, in which the gelling agent comprises agarose, agar, alginate, gellan, carrageenan, pectin, chitosan, chitin, gelatin or a mixture thereof.

19. A dental shaped body obtainable by the process according to claim 1.

20. A process of using a suspension of a zirconium oxide starting material as dental material, for the preparation of a dental shaped body, wherein the suspension comprises a gelling agent.
